(19) United States
(12) Patent Application Publication
(10) Pub. No.: US 2022/0351841 A1
(43) Pub. Date: Nov. 3, 2022

(54) INTERACTIVE MULTI-FACTOR PHYSICIAN SIGNATURE SYSTEM

(71) Applicant: George Pollack, Boca Raton, FL (US)

(72) Inventor: George Pollack, Boca Raton, FL (US)

(21) Appl. No.: 17/245,915

(22) Filed: Apr. 30, 2021

(51) Int. Cl. G16H 40/20; G06Q 10/10; G16H 10/60; H04L 29/06; G06F 21/60

(52) U.S. Cl. CPC G16H 40/20; G06Q 10/105; G16H 10/60; H04L 63/08; G06F 21/604; G16H 20/10

(57) ABSTRACT

A physician order system is disclosed including at least one computing device in operable connection with a network; a memory that stores computer-executable components; a processor that executes the computer-executable components stored in the memory, wherein the computer-executable components may include an application in communication with the computing device, the application comprising at least one module for permitting user access, user function management, user permissions, or data management; and wherein the application is constructed and arranged to permit a physician to execute a physician's order via a multi-factor encrypted compliant digital authentication and to transmit the executed physician's order to a recipient on a network.

### TECHNICAL FIELD

The embodiments generally relate to the field of electronic medical records and more specifically relate to electronically generating and processing physician's orders and prescriptions.

### BACKGROUND

Communication between physicians and other healthcare professionals is critically important in the practice of medicine to ensure a standard of care is maintained. Historically, orders are generated by physicians by writing an order on a chart for hospitalized patients, or by writing a prescription for outpatient services. Referrals are similarly communicated to a home care agency (e.g., a home health agency, durable medical equipment supplier, specialty pharmacy, or other home care industry provider).

There are two types of electronic medical record computer systems: the standalone system and the total home care agency system. The standalone system is an independent computer system that regulates operational tasks in the healthcare industry between two departments within the agency. The total home care agency system is similar in operation to the standalone system but has been incorporated into a computer network that interconnects all departments of the agency.

Advances have been made in health Insurance Portability and Accountability Act (HIPAA) compliant connectivity allowing physicians to communicate electronically with patients and healthcare professionals alike. These systems help to avoid delays in obtaining and executing orders, prescriptions, or re-certifications.

### SUMMARY

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments disclosed herein provide for a physician order system comprising an application provided on a computing device or accessible through a web-based portal. The application includes an interface to provide a means for a physician to execute a physician order and transmit the executed physician order to a recipient via a network.

A physician order system may include at least one computing device in operable connection with a network; a memory that stores computer-executable components; a processor that executes the computer-executable components stored in the memory, wherein the computer-executable components may include an application in communication with the computing device, the application including at least one of a module for permitting user access, user function management, user permissions, or data management; and wherein the application may be constructed and arranged to permit a physician to execute a physician's order via a multi-factor encrypted compliant digital authentication and to transmit the executed physician's order to a recipient on a network.

A physician order system that may include at least one computing device in operable connection with a network; a memory that stores computer-executable components; a processor that executes the computer-executable components stored in the memory, wherein the computer-executable components may include: an application in communication with the computing device, the application including at least one of a module for permitting user access, user function management, user permissions, or data management; a graphical user interface including a dashboard module; a provider list module; a business type user list module; an invitation module; and wherein the application may be constructed and arranged to permit a physician to execute a physician's order via a multi-factor encrypted compliant digital authentication and to transmit the executed physician's order to a recipient on a network.

A physician order system that may include at least one computing device in operable connection with a network; a memory that stores computer-executable components; a processor that executes the computer-executable components stored in the memory, wherein the computer-executable components may include a user registration module constructed and arranged to facilitate user type registration, identify unique users and prevent duplicate users, and notify an administrator of duplicate users; a user management module constructed and arranged to allow an administrator to allow or deny user registration; and a searchable data management module constructed and arranged to store, catalog, organize, retrieve, and communicate information.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure, according to some embodiments described herein;

FIG. 2 illustrates a block diagram of the network infrastructure, according to some embodiments described herein;

FIG. 3 illustrates a block diagram of the server engine and associated modules, according to some embodiments described herein;

FIG. 4 illustrates a flowchart of a use case diagram according to some embodiments described herein;

FIG. 5 illustrates a flowchart of a use case diagram according to some embodiments described herein;

FIG. 6 illustrates a flowchart of a use case diagram according to some embodiments described herein;

FIG. 7 illustrates a flowchart of a use case diagram according to some embodiments described herein;

FIG. 8 illustrates a flowchart of a use case diagram according to some embodiments described herein;

FIG. 9 illustrates a flowchart of a use case diagram according to some embodiments described herein;

FIG. 10 illustrates a flowchart of a use case diagram according to some embodiments described herein;

FIG. 11 illustrates a flowchart of a use case diagram according to some embodiments described herein;

FIG. 12 illustrates a graphical user interface according to some embodiments described herein;

FIG. 13 illustrates a graphical user interface according to some embodiments described herein;

FIG. 14 illustrates a graphical user interface according to some embodiments described herein;

FIG. 15 illustrates a graphical user interface according to some embodiments described herein;

FIG. 16 illustrates a graphical user interface according to some embodiments described herein;

FIG. 17 illustrates a graphical user interface according to some embodiments described herein;

FIG. 18 illustrates a graphical user interface according to some embodiments described herein;

FIG. 19 illustrates a graphical user interface according to some embodiments described herein; and

FIG. 20 illustrates a graphical user interface according to some embodiments described herein.

FIG. 21 illustrates a flow diagram illustrating an example process of a system enabling a physician's execution of an order via multi-factor encrypted compliant digital authentication.

### DETAILED DESCRIPTION

The specific details of the single embodiment or variety of embodiments described herein are to the described system and methods of use. Any specific details of the embodiments are used for demonstration purposes only and no unnecessary limitations or inferences are to be understood from there.

It is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, apparatus, and/or computer program product at any possible technical detail level of integration. A computer program product can include, among other things, a computer-readable storage medium having computer-readable program instructions thereon for causing a processor to carry out aspects of the present disclosure.

In general, the embodiments described herein relate to an interactive multi-factor encrypted physician signature system for use on a mobile computing device or web-based app. The system discloses a HIPAA compliant system which allows for physicians or other authorized users to receive orders or prescriptions through a plurality of portals originating from healthcare providers or home care agencies. Physicians may then view these orders or prescriptions on a single user interface, allowing the physician to securely authenticate and sign the orders/prescriptions using an encryption mode. The system further contemplates having an alert notification transmitted to the prescribed physician when a new order has been received and allowing for payment when orders have been signed and transmitted to the pre-determined healthcare provider or home care agency. The system may include a memory module which will store the signed orders or prescriptions, and which may be viewable by patients, physicians, or others.

As used herein, the term “user(s)” may refer to physicians, physician office staff, home healthcare agency personnel, or other persons in the healthcare field or patients in the healthcare field.

As used herein, “GUI” may refer to any graphical user interface that includes at least one interactive component between a user and the application. A GUI may include a plurality of fillable fields, clickable buttons, database displays, or the like. A GUI maybe adaptable for use on several devices such as computers, phones, smart devices, tablets, laptops, televisions, or the like.

In this disclosure, terms “store,” “storage,” “data store,” “data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random-access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

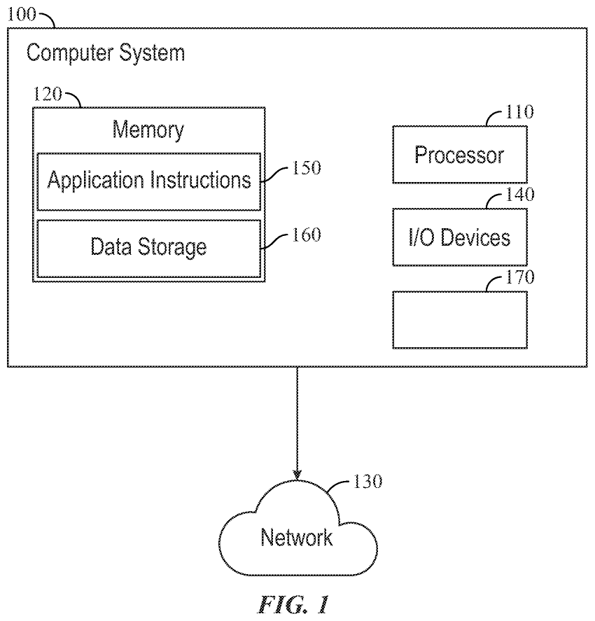

Referring to FIG. 1, a computer system 100 may include memory 120, a processor 110, input/output devices 140, and a network interface 170 constructed and arranged to facilitate communication with a network 130. The memory 120 may include computer-readable application instructions 150, configured to implement certain embodiments described herein, and a database 160, comprising various data accessible by the application instructions 150. In some embodiments, the steps and actions of the application instructions 150 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 150 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 150 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 150 can be downloaded to a computing/processing device from a computer readable storage medium, or to an external computer or external storage device via a network 130. A network adapter card or network interface in each computing/processing device receives computer readable program instructions from the network and forwards the computer readable application instructions 150 for storage in a computer readable storage medium within the respective computing/processing device.

In some embodiments, the computer system 100 includes a network interface 170 to communicate with a network 130. In some embodiments, the network interface 170 is configured to allow data to be exchanged between the computer system 100 and other devices attached to the network 130, such as other computer systems, or between nodes of the computer system 100. In various embodiments, the network interface 170 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

In some embodiments, the network 130 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 130 can comprise copper transmission cables, optical transmission fibers, wireless transmission, routers, firewalls, switches, gateway computers and/or edge servers. The network 130 can represent a single network or multiple networks. In some embodiments, the network 130 used by the various devices of system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

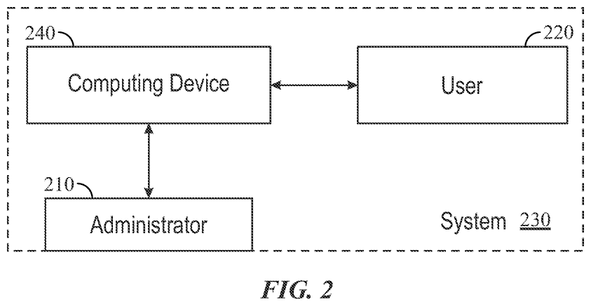

In reference to FIG. 2, an exemplary network infrastructure is illustrated having an administrator 210 who can create, delete, activate, or deactivate users of the system, in addition to performing other administrative tasks. A user 220 operates the computing device 240 to generate, edit, receive, or otherwise interact with physician orders or other documents created using the system which may require a signature or other authorization. The system 230 utilizes at least one computing devices 240 to permit a user interface to allow a physician to execute a signature on a physician order or other form of communication.

In some embodiments, the home care agency or care provider (i.e., a physician, or other medical personnel) registers the patient with login credentials, including a name, date of birth, photograph, username/email, and password. A care provider registers with their name, credentials, contact information, and practice information.

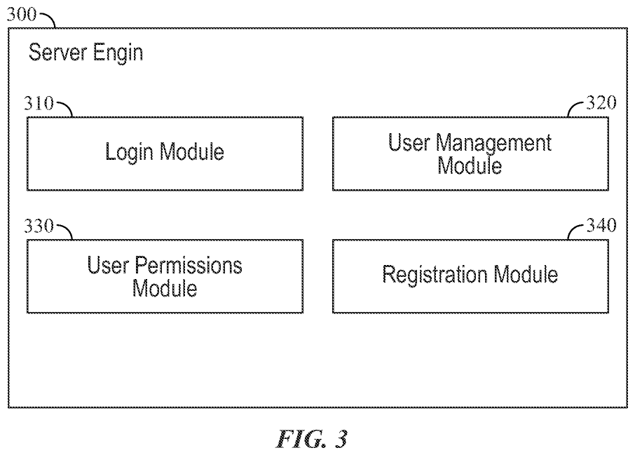

In reference to FIG. 3, the server engine 300 and modules are illustrated. A login module 310 authorizes users to log in to the application and performs processes for password recovery, or other login credential changes. The login module 310 may include captcha functionality to determine whether the user is human or a computer. The login module 310 or user access module may include additional security measures such as electronic authentication, including multi-factor authentication or two factor authentications. Additional security features and security measures may be implemented throughout the system as will be described herein.

The user management module 320 may be utilized by an administrator to invite users such as patients, providers, and businesses as well as creating roles for each new or existing user. The user management module 320 may allow the creation, deletion, activation, or deactivation of other users and may receive user information, where the user is a patient, such as last name, middle name or initial, date of birth, photograph, username, payment information, email, and/or password.

A user permissions module 330 provides the functionality to the administrator to provide access for the users to one or more of the plurality of modules described herein. The user permissions module 330 may also allow the administrator to view, modify, and administer user permission settings within the applications.

A user registration module 340 is provided to intake and process user registration information, including patient credentials and payment information. A user may not be able to login to the application until an administrator verifies the patient account. The user registration module 340 may be utilized by the administrator to invite users such as patients, providers, and businesses as well as creating roles for each new or existing user. Roles may be defined as administrative, business type, patients, and providers. The user registration module 340 may require the administrator to verify patient details prior to allowing the patient access to the system.

Where the user is a provider, the user registration module 340 may receive provider information such as physicians last name, middle name or initial, first name, suffix (such as MD, DO, DPM, DNP, ARNP, NP & PA), phone number, email address, mobile number, office number, office fax, address, administrator or key contact person, or other relevant contact information. The user registration module 340 may also receive information regarding payment methods such as credit card details, banking information, or the like. The user registration module 340 may require the administrator to verify provider details prior to allowing the provider access to the system.

Where the user is a business, the user registration module 340 may receive information such as an administrator in the business or key contact person, the business name, or other relevant contact information such as office phone number, office fax, office address, or a variety of other means of contacting the business or individuals within the business. Where the user is a business, the system may allow the business to manage patient registration.

A physician may utilize the system to sign a physician order. The system may utilize a two-factor authentication, or multi factor authentication system or similar means of security known in the arts. Once the physician signs in, the physician may receive a numeric code on their computing device via an email or a text message. The system will then transmit the physician order file to a pending section within the user interface for the physician to sign. The pending section allows the physician to select the order document, review the document, communicate any changes or amendments that must be made to the document, and sign the document for electronic transmission to the recipient.

In some embodiments, the system will transmit an alert to the physician to remind the physician that the service can be billed to a health plan or other medical billing service.

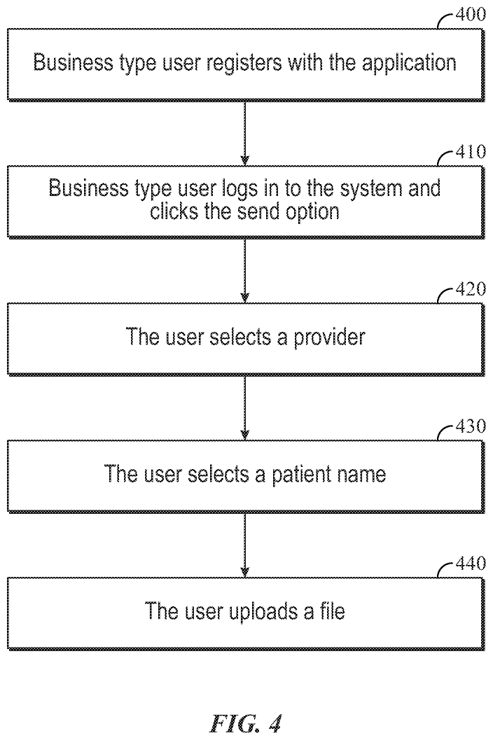

In reference to FIG. 4, a flowchart of a use case diagram includes a business type user 400 which logs in to send a file in step 410 and clicks the send option in step 410. The sent file then allows the user to select a provider in step 420, a patient name in step 430, an order type in step 440, and upload a file in step 440.

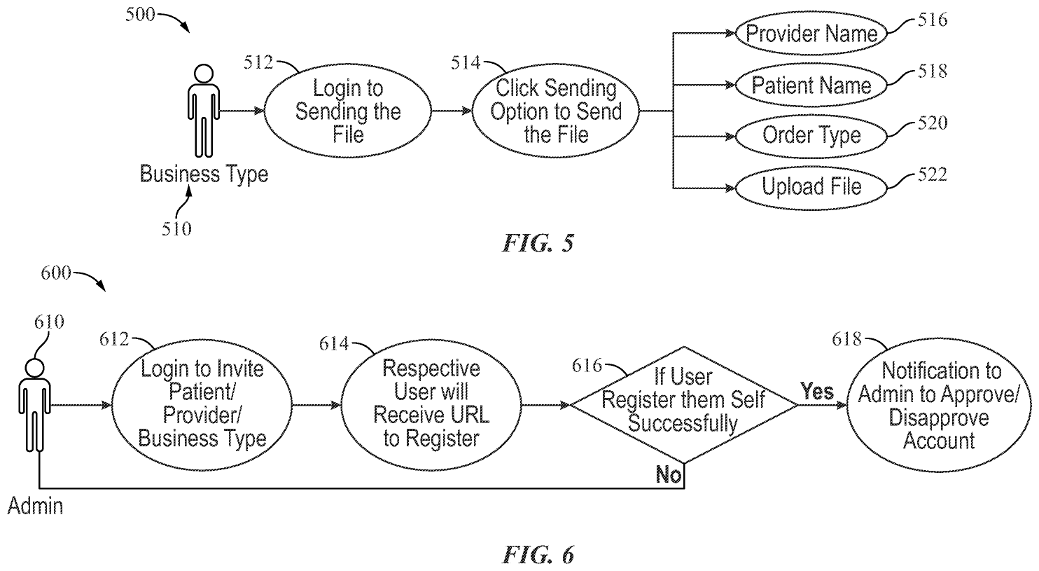

In reference to FIG. 5, a flowchart of a use case diagram includes a system 500 wherein a business type user 510 may log into the system 512 to submit, send, or upload a file such as a prescription order. The business type user 510 may submit the order 514 via a confirmation action such as a clickable “SUBMIT” button within a GUI of the system 500. The submission of the order 514 may include providing relevant information such as provider name 516, patient name 518, order type 520, and attached file or order 522.

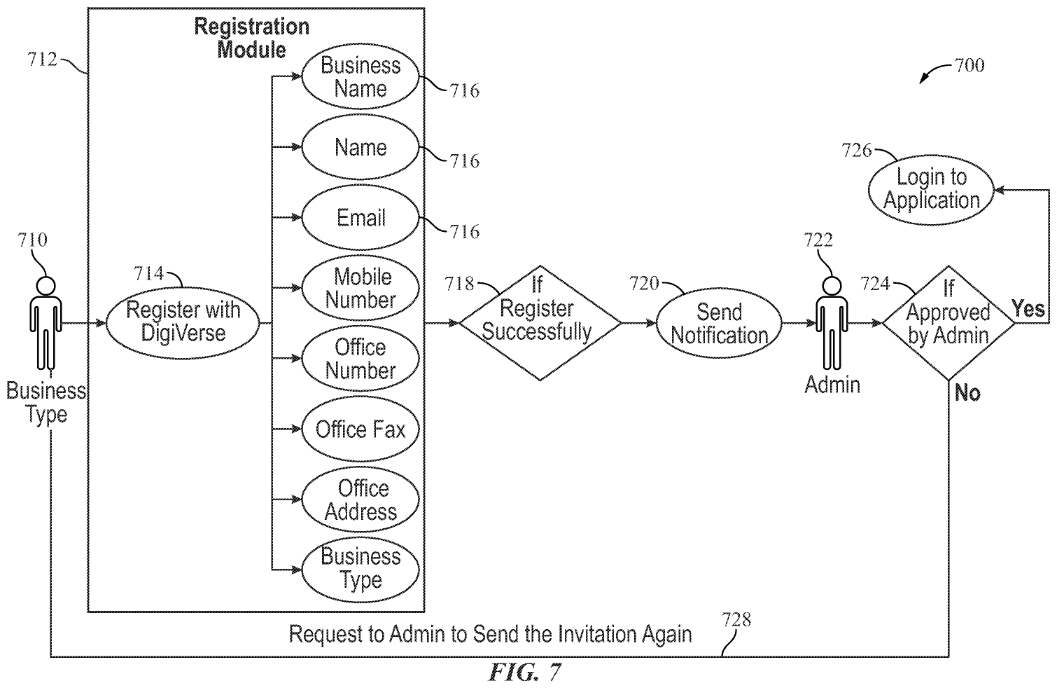

In reference to FIG. 6, a flowchart of a use case diagram includes a system 600 wherein an administrator 610 may log in to the system 612 to invite a user such as a patient, or provider, or business type by sending a unique URL via email. The user may receive the invitation 614 and register 616 in the system via the URL. Where the user has successfully registered 616 the system may automatically generate a notification to the administrator to approve or disapprove of the account registration. Where the user has unsuccessfully registered 616 the system may automatically generate a notification to the administrator of such unsuccessful registration.

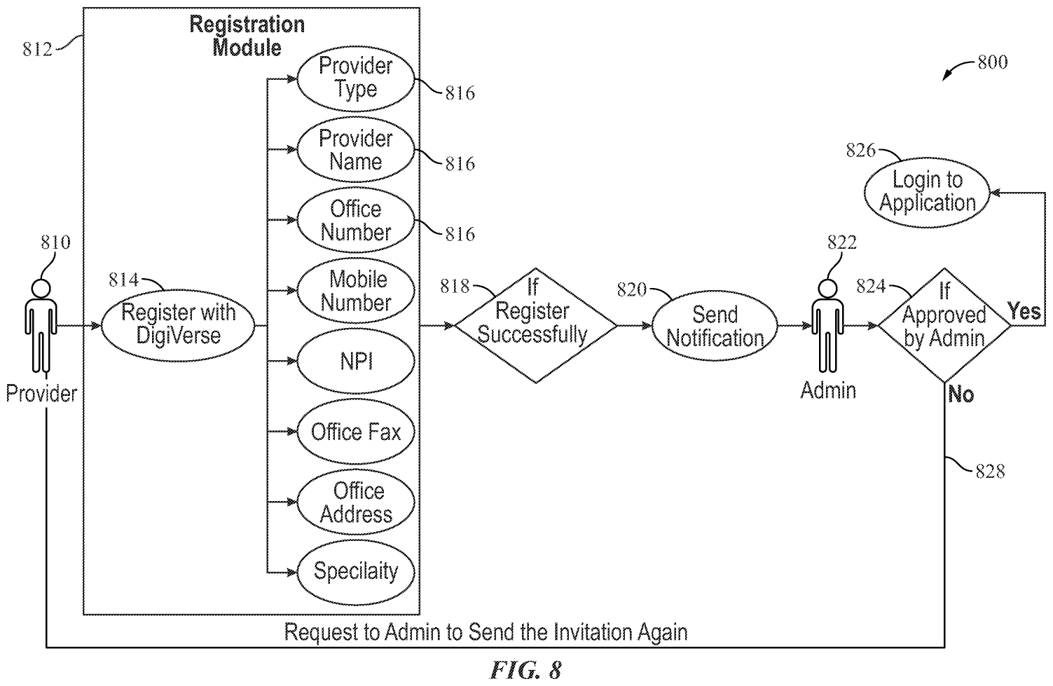

In reference to FIG. 7, a flowchart of a use case diagram includes a system 700 wherein a user 710 may log into the system 700 and more specifically the user registration module 712. The user 710 may be a business. The user 710 may enter user information 716 such as a business name, username, email, phone number, business phone number, fax number, or physical address into a fillable form such as a web based or internet browser-based portal to register 714 with the system 700. Where the user has successfully registered 718 the system may automatically generate a notification 720 to the administrator 722 to approve or disapprove of the account registration 724. If the administrator 722 approves of the account registration 724, the user will be permitted to log into the application 726 via a temporary password. A user may be required to modify or change their password, add new security measures such as security questions, and may be required to subscribe to a secure payment gateway when logging into the system for the first time. If the administrator 722 disapproves of the account registration 724 the user 710 may request a new invitation 728 or the system 700 may automatically generate a new invitation 728 to be sent to the user 710.

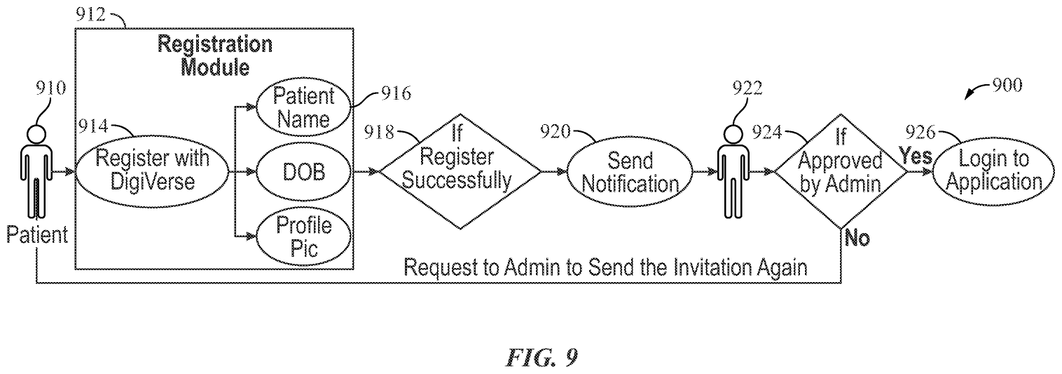

In reference to FIG. 8, a flowchart of a use case diagram includes a system 800 where in a user 810 may log into the system 800 and more specifically the registration module 812. The user 810 may be a healthcare provider. The user 810 may enter user information 816 such as provider type, provider name, office number, phone number, fax number, or physical address into a fillable form such as a web based or internet browser-based portal to register 814 with the system 800. Where the user 810 has successfully registered 818 the system may automatically generate a notification 820 to the administrator 822 to approve or disapprove of the account registration 824. If the administrator 822 approves of the account registration 824, the user will be permitted to log into the application 826 via a temporary password. A user may be required to modify or change their password, add new security measures such as security questions, and may be required to subscribe to a secure payment gateway when logging into the system for the first time period if the administrator 822 disapproves of the account registration 824 the user 810 may request a new invitation 828 or the system 800 may automatically generate a new invitation 828 to be sent to the user 810.

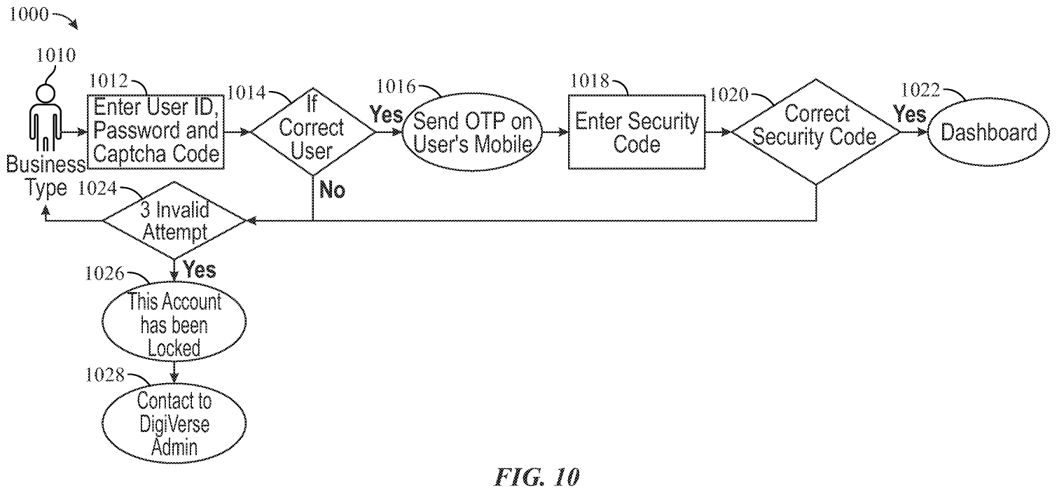

In reference to FIG. 9, a flowchart of a use case diagram includes a system 900 where a user 910 may log into the system 900 and more specifically the registration module 912. The user 910 maybe a healthcare patient. The user 910 may enter user information 916 such as patient name, date of birth, and picture into a fillable form such as a web based or Internet browser-based portal to register 914 with the system 900. Where the user 910 has successfully registered 918 the system may automatically generate a notification 920 to the administrator 922 after which the administrator 922 may approve or disapprove of the account registration 924. If the administrator 922 approves of the account registration 924, the user may be permitted to log into the application 926 via a temporary password. A user may be required to modify or change their password, add new security measures such as security questions, and may be required to subscribe to a secure payment gateway when logging into the system for the first time period if the Administrator 922 disapproves of the account registration 924 the user 910 may request a new invitation 928 or the system 900 may automatically generate a new invitation 928 to be sent to the user 910.

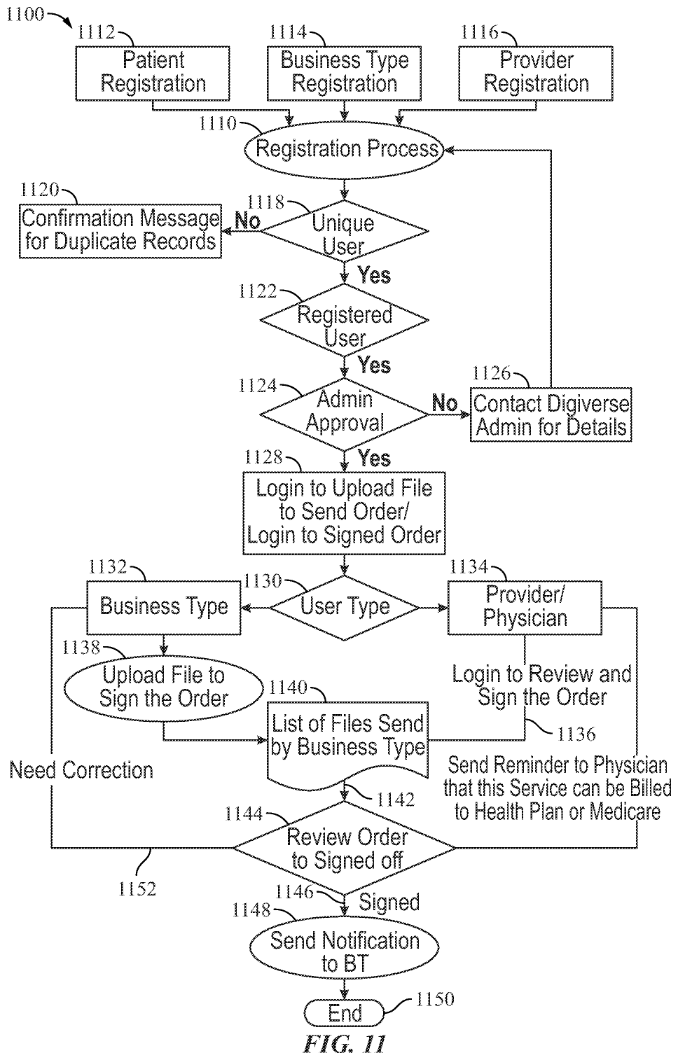

In reference to FIG. 10, a flowchart of a use case diagram includes a system 1000 Where a user 1010 may enter their user login information such as user ID, password, or the like 1012 and the entered credentials may be compared against system data 1014 as a security measure. If the entered credentials are match the credentials stored by the system 1000, a one-time password may be sent to the user 1010. The user 1010 may enter 1018 the one-time password into the system 1000 which may confirm that the one-time password is correct 1020. If the one-time password entered matches the credentials required by the system, the user may be provided access to the system. Three or more invalid attempts 1024 to enter user ID, password, or the one-time password may generate an automatic alert to an administrator 1028 or may temporarily lock the user account 1026.

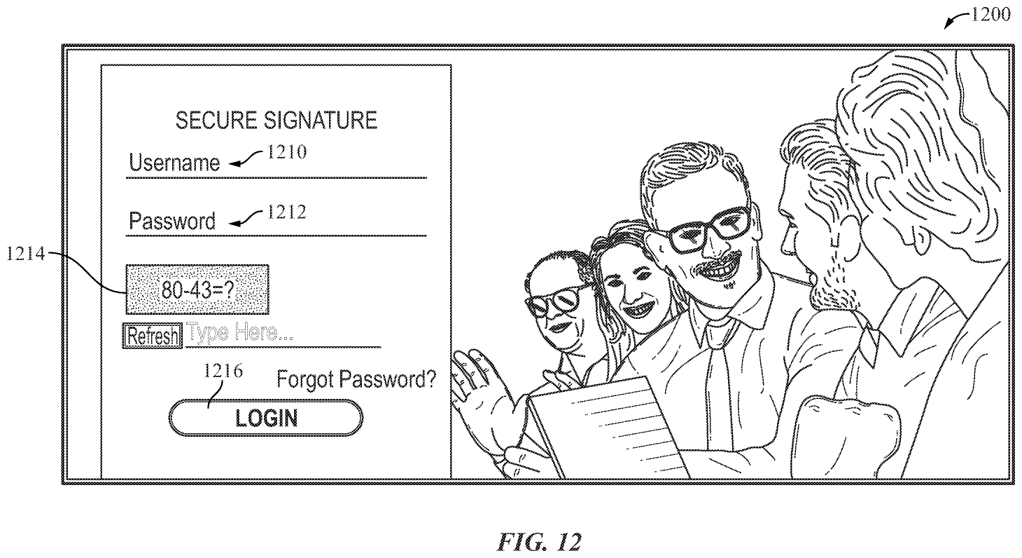

In reference to FIG. 11, a flowchart of a use case diagram includes a system 1100 That may include a registrations process 1110, that may include patient registration 1112, business type registration 1114, and provider registration 1116. The registration process 1110 may be constructed and arranged to identify unique users 1118 which may then become registered users 1122 according to the methods described previously. User registration determined not to be unique may provide an alert confirming duplicate records 1120 and may prevent registration of duplicate users. Administrator approval 1124 of user registration may be denied 1126 and a user attempting to register may be requested to attempt the registration process 1110 again. Successful administrator approval 1124 may allow a user access to the system 1100 and may allow a user certain functionality's based upon user type. Business type users 1132 may be permitted to upload files 1138 for signature by a physician or health care provider. Uploaded files 1138 may be provided to a physician or health care provider for review and signature. A provider or physician type user 1134 may be permitted to log in to review uploaded files 1136. The system 1100 may be constructed and arranged to send a notification or reminder to a provider or physician regarding health care insurance program details 1142. A database module maybe constructed and arranged to list files uploaded by business type users and which may be catalogued and searchable 1140. A provider or physician type user 1134 may review uploaded files such as prescription orders for accuracy and if correction is needed 1152 may return the file to the business type user 1132. A provider or physician type user 113 4 may review uploaded files for accuracy and if no correction is required, the provider or physician type user 1134 may electronically sign the file as needed. The electronically signed file may then be returned to the business type user 1132.

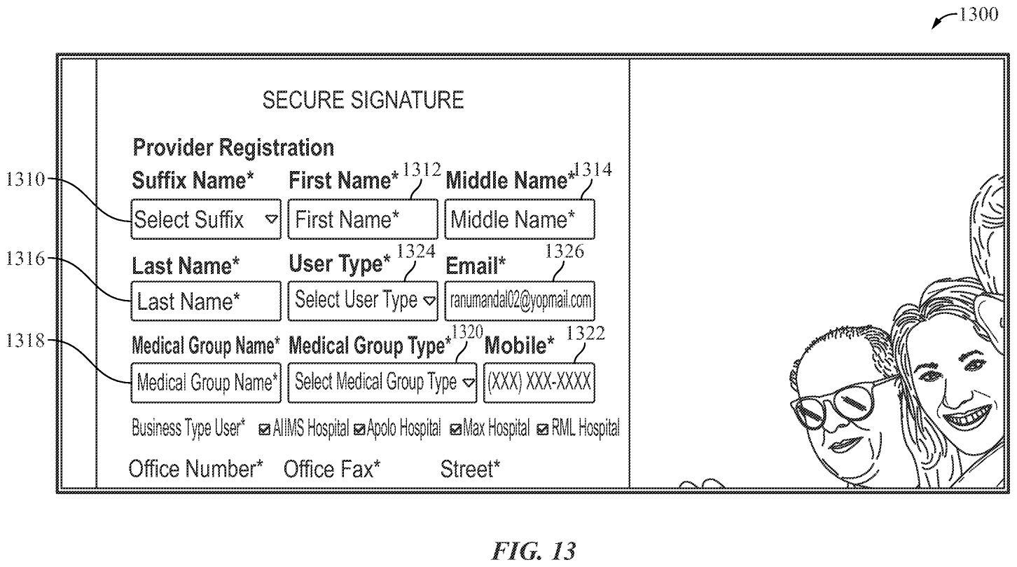

FIG. 12 depicts a GUI including a login module 1200 to permit one or more users to login to the application. The GUI 1200 may include a fillable form portion for entering a username 1210, password 1212, and may also include a security measure 1214 such as a security measure including captcha functionality. The GUI 1200 may include a clickable button 1216 to submit user entered information. The GUI 1200 may require additional information from the user.

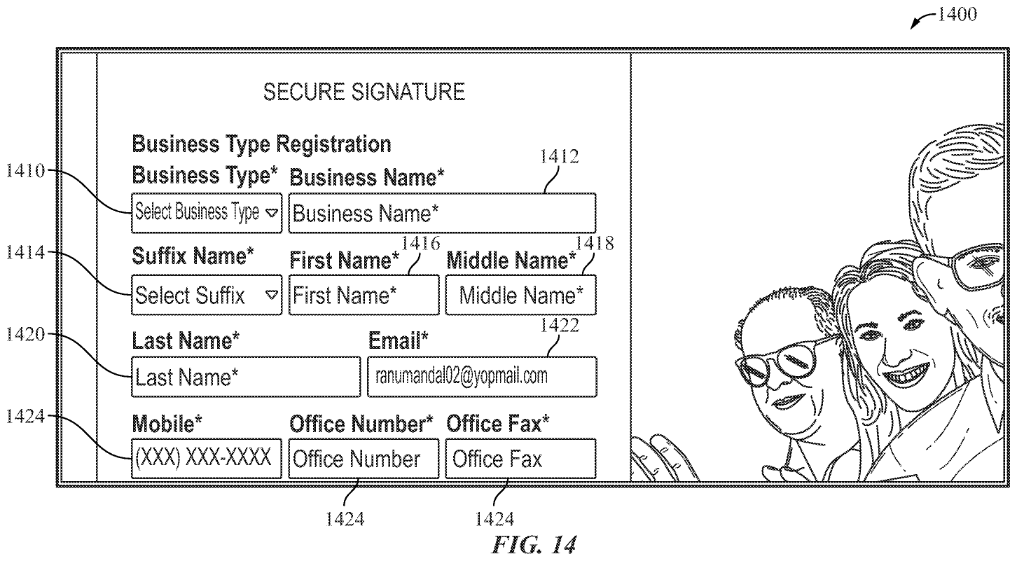

FIG. 13 depicts a GUI including a provider registration module 1300 to permit a health care provider to register as a user of the application. The GUI 1300 may include a plurality of syllable fields to record and store provider information. The fillable fields may request information such as, but not limited to, provider suffix 1310, first name 1312, middle name 1314, last name 1316, user type 1324, user email address 1326, medical group name 1318, medical group type 1320, phone contact information, and other various information as needed. The GUI 1300 may require additional information from the user.

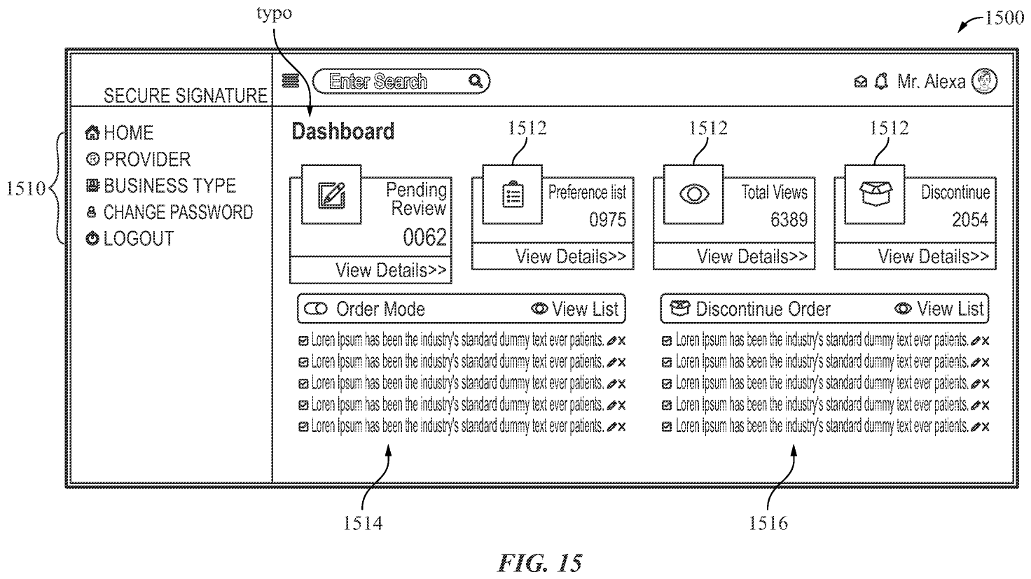

FIG. 14 depicts a GUI including a business type registration module 1400 to permit a business type to register as a user of the application. The GUI 1400 may include a plurality of fillable fields to record and store business type information. The full fields may request information such as come up but not limited to, business type 1410, business name 1412, suffix 1414, first name 1416, middle name 1418, last name 1420, contact email address 1422, and other contact information 1424. The GUI 1400 may require additional information from the user.

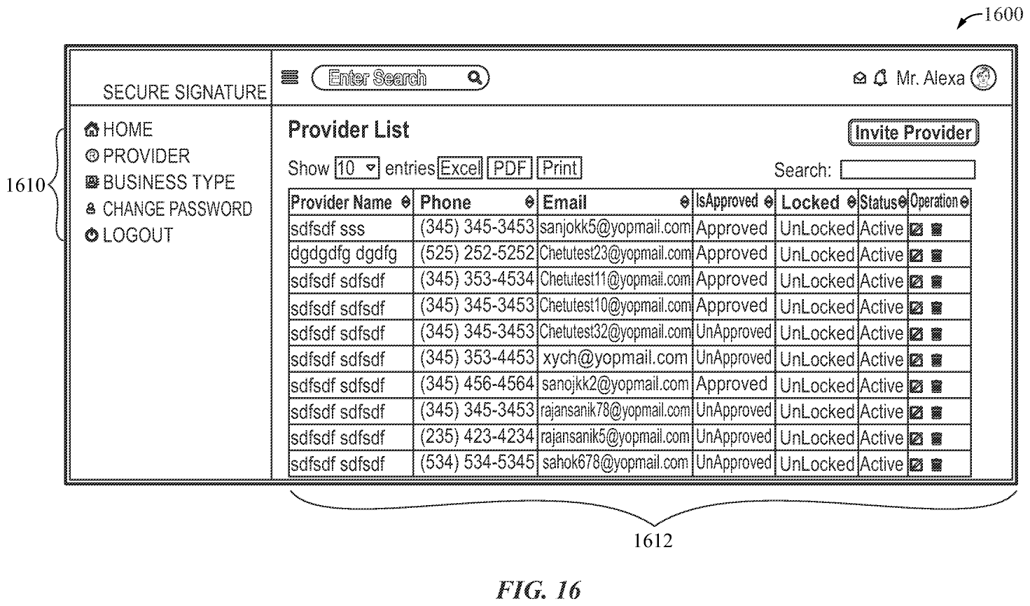

FIG. 15 depicts a GUI including a user dashboard module 1500 that may include a navigation menu of clickable buttons or links 1510 which may provide a user with information relating to their health care provider, business type, or other information including administrative settings such as changing username, password, contact information, or the like. The dashboard module 1500 may be constructed and arranged to display relevant information 1512 regarding information rich requires review, completed tasks, cancelled orders or tasks, total views, or the like. The dashboard module 1500 may be constructed and arranged to display pending orders 1514 and the discontinued orders 1516 as catalogued in a readable database module stored by the system.

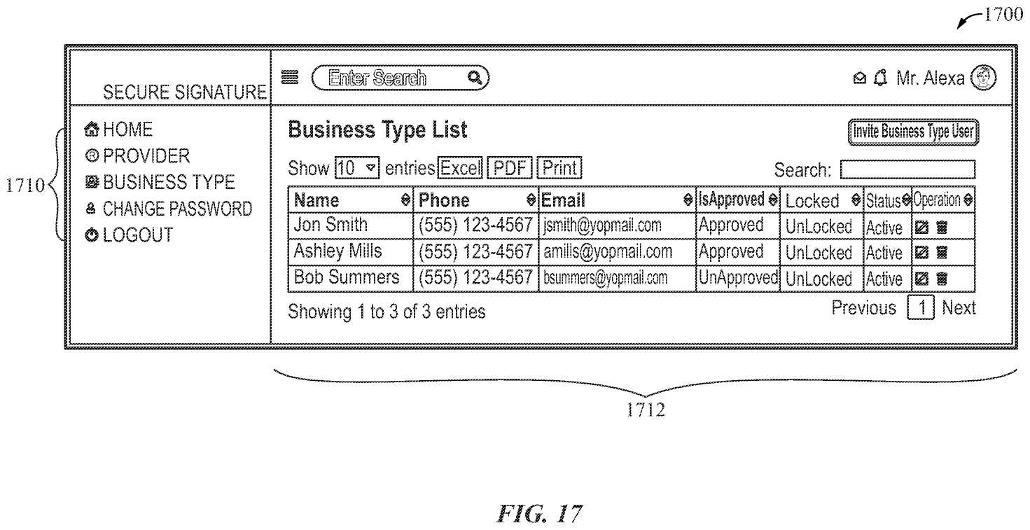

FIG. 16 depicts a GUI including a provider list module 1600 that may include a navigation menu 1610. The provider list module 1600 may be constructed and arranged to display provider information 1612 as catalogued in a readable database stored by the system. The provider list module 1600 may allow an administrator to approve or disapprove, lock, or unlock, activate, or deactivate, or edit provider information.

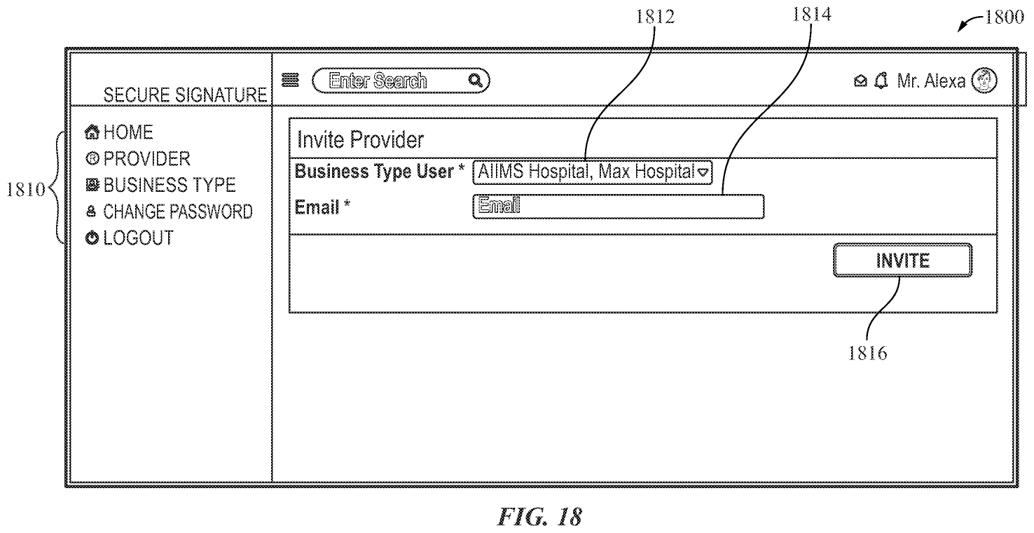

FIG. 17 depicts a GUI including a business type user list module 1700 that may include a navigation menu 1710. The business type user list module 1700 may be constructed and arranged to display information 1712 regarding business types as catalogued in a readable database stored by the system. The business type user list module 1700 may allow an administrator to approve or disapprove, lock, or unlock, activate, or deactivate, or edit provider information.

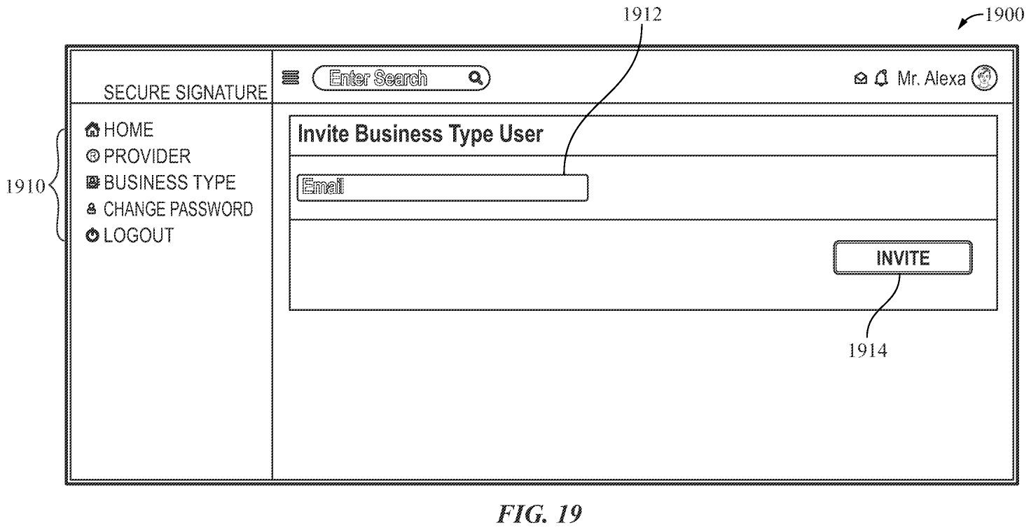

FIG. 18 depicts a GUI including a provider invitation module 1800 the may including navigation menu 1810. The provider invitation module 1800 may include fillable field such as provider type 1814 and contact information 1812. The provider invitation module 1800 may include a clickable button 1816 that maybe constructed and arranged to dispatch an invitation to the submitted provider 1814 and contact information 1812 such that a provider may be invited to register for the system.

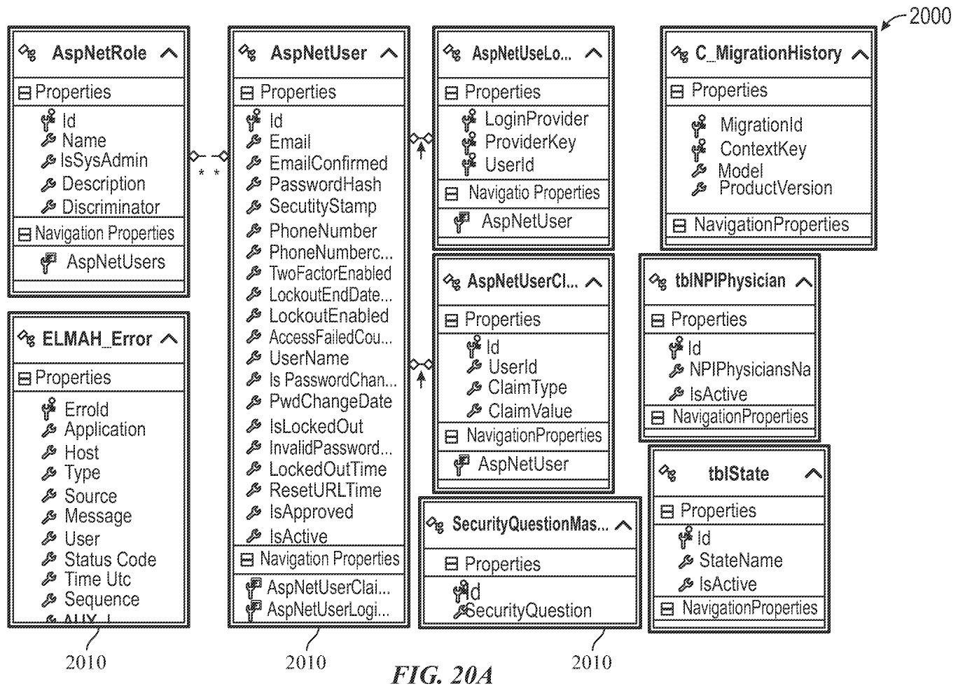

FIG. 19 depicts a GUI including a business type invitation module 1900 that may include a navigation menu 1910. The business type invitation module 1900 may include a fillable field 1912 such as contact information such as an email address. The business type invitation module 1900 may include a clickable button 1914 that may be constructed and arranged to dispatch an invitation to the submitted business type contact information 1912 such that a business type may be invited to register with the system.

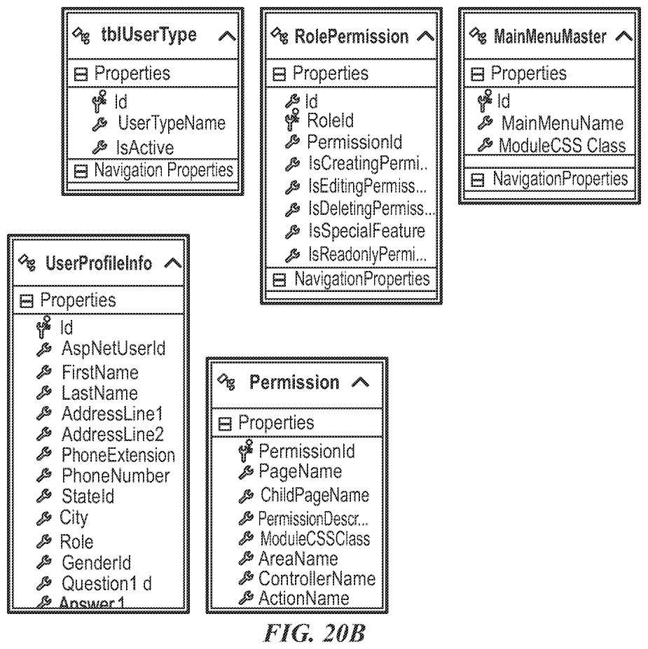

FIG. 20 depicts a GUI including a database module 2000 that may be constructed and arranged to store, catalog, organized, and display information 2010 submitted to the system.

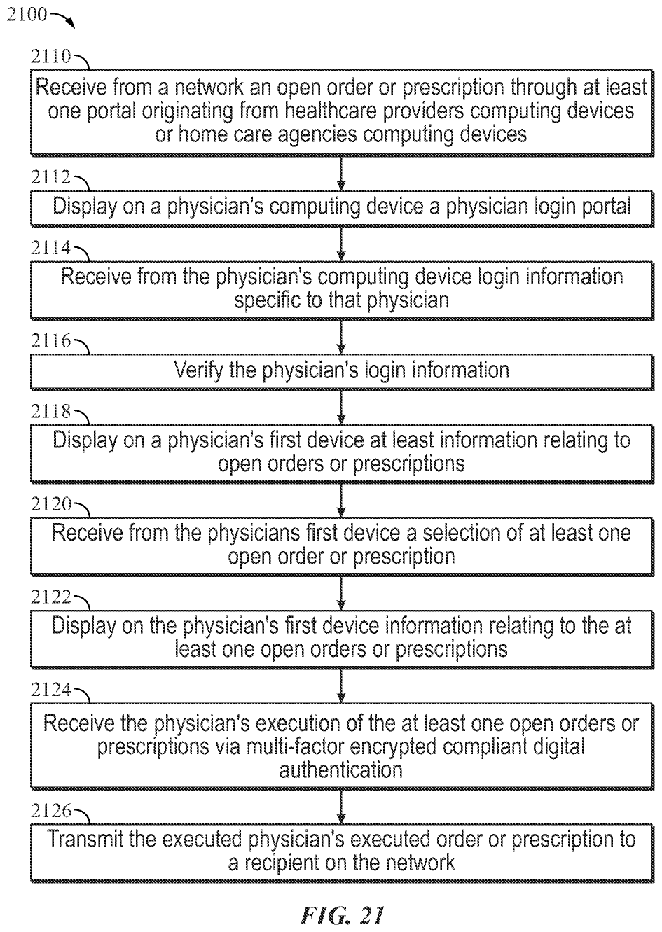

FIG. 21 is a flow diagram illustrating an example process 2100 of the system enabling a physician's execution of an order via multi-factor encrypted compliant digital authentication. Step 2110 may include receiving a plurality of orders or prescriptions from a healthcare provider or home care agency or the like. The orders or prescriptions may originate from a computing device such as a computer, laptop, mobile device, or the like. The orders or prescriptions may be communicated via a local area network such as in a hospital or via an internet network such as a server-based network or similar. Step 2112 may include providing and displaying a login portal for a physician or healthcare provider on a computing device such as a computer, laptop, mobile device, or the like. The login portal may require user information such as, but not limited to, username and password. The login portal may include captcha functionality as well as other secure login means. Step 2114 may include receiving the user login information such as, but not limited to, username and password. The user may enter said information via an input device such as a touchscreen keypad, a keyboard, voice command, or the like. Step 2116 may include the system verifying the authenticity of the user-entered user information. Verification may include comparing the user-entered user information to stored or known user information collected during an earlier registration process. Where user information cannot be verified, the system may prevent a user from logging into the system. Upon verification, Step 2118 may include displaying a plurality of received orders or prescription from step 2110 on the user's computing device. Displaying the received order or prescription may include providing additional information including, but not limited to, patient information such was name, date of birth, contact information, medical history, or the like. Step 2120 may include receiving input from the user including a selection of at least one order or prescription. The input may be selection of an order or prescription by selecting said order or prescription via computer mouse click, touchscreen selection, voice command, keyboard selection, or other various means of providing input to the computing device. Step 2122 may include providing and displaying on the user's computing device information associated with the selected order or prescription including, but not limited to, prescription and order information, patient information such as name, date of birth, contact information, medical history, or the like. Step 2124 may include providing a means for a receiving a physician's signature or execution of the order or prescription. Step 2124 may include multi-factor encrypted compliant digital authentication of the physician's signature or execution as well as other security measures such that the execution of the order or prescription is HIPAA compliant. Step 2126 may include transmitting the executed order or prescription to a recipient on the network. The transmission may be made on a local area network or through a secure transmission via the internet. The recipient may be another healthcare provider, physician, physician's staff, pharmacy, hospital, or other healthcare related individual or business.

The following description of variants is only illustrative of components, elements, acts, products, and methods considered to be within the scope of the invention and are not in any way intended to limit such scope by what is specifically disclosed or not expressly set forth. The components, elements, acts, products, and methods as described herein may be combined and rearranged other than as expressly described herein and are still considered to be within the scope of the invention.

According to variation 1, a physician order system may include at least one computing device in operable connection with a network; a memory that stores computer-executable components; a processor that executes the computer-executable components stored in the memory, wherein the computer-executable components may include an application in communication with the computing device, the application including at least one module for permitting user access, user function management, user permissions, or data management; and wherein the application may be constructed and arranged to permit a physician to execute a physician's order via a multi-factor encrypted compliant digital authentication and to transmit the executed physician's order to a recipient on a network.

Variation 2 may include the physician order system as set forth in variation 1 wherein the user access module may be constructed and arranged to permit one or more users to login to the application.

Variation 3 may include the physician order system as set forth in any of variations 1 through 2 wherein the user management module may be constructed and arranged to permit an administrator to manage a plurality of user functions including at least one of dispatching invitations to users or creating user roles.

Variation 4 may include the physician order system as set forth in any of variations 1 through 3 wherein the user permission module may be constructed and arranged to permit the administrator to control user permissions including at least one of viewing, modifying, or administering settings.

Variation 5 may include the physician order system as set forth in any of variations 1 through 4 wherein the data management module may be constructed and arranged to store, catalog, organize, retrieve, and communicate information.

Variation 6 may include the physician order system as set forth in any of variations 1 through 5 further including a user registration module constructed and arranged to intake and process user registration information and permit an administrator to verify a submitted user registration.

Variation 7 may include the physician order system as set forth in any of variations 1 through 6 wherein the application further may include a graphical user interface including a dashboard module.

Variation 8 may include the physician order system as set forth in any of variations 1 through 7 further including a provider list module.

Variation 9 may include the physician order system as set forth in any of variations 1 through 8 further including a business type user list module.

Variation 10 may include the physician order system as set forth in any of variations 1 through 9 further including an invitation module.

Variation 11 may include a physician order system that may include at least one computing device in operable connection with a network; a memory that stores computer-executable components; a processor that executes the computer-executable components stored in the memory, wherein the computer-executable components may include: an application in communication with the computing device, the application including at least one module for permitting user access, user function management, user permissions, or data management; a graphical user interface including a dashboard module; a provider list module; a business type user list module; an invitation module; and wherein the application may be constructed and arranged to permit a physician to execute a physician's order via a multi-factor encrypted compliant digital authentication and to transmit the executed physician's order to a recipient on a network.

Variation 12 may include the physician order system as set forth in any of variation 11 wherein the user access module may be constructed and arranged to permit one or more users to login to the application.

Variation 13 may include the physician order system as set forth in any of variations 11 through 12 wherein the user management module may be constructed and arranged to permit an administrator to manage a plurality of user functions including at least one of dispatching invitations to users or creating user roles.

Variation 14 may include the physician order system as set forth in any of variations 11 through 13 wherein the user permission module may be constructed and arranged to permit the administrator to control user permissions including at least one of viewing, modifying, or administering settings.

Variation 15 may include the physician order system as set forth in any of variations 11 through 14 wherein the data management module may be constructed and arranged to store, catalog, organize, retrieve, and communicate information.

Variation 16 may include the physician order system as set forth in any of variations 11 through 15 further including a user registration module constructed and arranged to intake and process user registration information and permit an administrator to verify a submitted user registration.

Variation 17 may include a physician order system that may include at least one computing device in operable connection with a network; a memory that stores computer-executable components; a processor that executes the computer-executable components stored in the memory, wherein the computer-executable components may include a user registration module constructed and arranged to facilitate user type registration, identify unique users and prevent duplicate users, and notify an administrator of duplicate users; a user management module constructed and arranged to allow an administrator to allow or deny user registration; and a searchable data management module may be constructed and arranged to store, catalog, organize, retrieve, and communicate information.

Variation 18 may include the physician order system as set forth in variation 17 and may further include a user permission module; and wherein the user type may be at least one of a business types, healthcare provider, physician, or patient and wherein each user type may be allowed permissions within the system as established by the user permission module.

Variation 19 may include the physician order system as set forth in any of variations 17 through 18 wherein the user permission module may be constructed and arranged to permit business type users to upload files for signature by a physician or health care provider and healthcare providers and physician users are permitted to review, upload files, and execute orders.

Variation 20 may include the physician order system as set forth in any of variations 17 through 19 wherein the application may be constructed and arranged to permit a physician to execute a physician's order via a multi-factor encrypted compliant digital authentication and to transmit the executed physician's order to a recipient on a network.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general-purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the flowchart and block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for anyone of the elements in the claims below or that a single element can be substituted for two or more elements in a claim. Although elements can be described above as acting in certain combinations, and even initially claimed as such, it is to be expressly understood that one or more elements from a claimed combination can, in some cases, be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible considering the above teachings without departing from the following claims.

What is claimed is:

1. A physician order system comprising:
at least one computing device in operable connection with a network;
a memory that stores computer-executable components;
a processor that executes the computer-executable components stored in the memory, wherein the computer-executable components comprise:
an application in communication with the computing device, the application comprising at least one of a module for permitting user access, user function management, user permissions, or data management; and
wherein the application is constructed and arranged to permit a physician to execute a physician's order via a multi-factor encrypted compliant digital authentication and to transmit the executed physician's order to a recipient on a network.

2. The physician order system of claim 1, wherein the user access module is constructed and arranged to permit one or more users to login to the application.

3. The physician order system of claim 1, wherein the user management module is constructed and arranged to permit an administrator to manage a plurality of user functions including at least one of dispatching invitations to users or creating user roles.

4. The physician order system of claim 1, wherein the user permission module is constructed and arranged to permit the administrator to control user permissions including at least one of viewing, modifying, or administering settings.

5. The physician order system of claim 1, wherein the data management module is constructed and arranged to store, catalog, organize, retrieve, and communicate information.

6. The physician order system of claim 1, further comprising a user registration module constructed and arranged to intake and process user registration information and permit an administrator to verify a submitted user registration.

7. The physician order system of claim 1, wherein the application further comprises a graphical user interface comprising a dashboard module.

8. The physician order system of claim 1, further comprising a provider list module.

9. The physician order system of claim 1, further comprising a business type user list module.

10. The physician order system of claim 1, further comprising an invitation module.

11. A physician order system, comprising:
at least one computing device in operable connection with a network;
a memory that stores computer-executable components;
a processor that executes the computer-executable components stored in the memory, wherein the computer-executable components comprise:
an application in communication with the computing device, the application comprising at least one of a module for permitting user access, user function management, user permissions, or data management;
a graphical user interface comprising a dashboard module;
a provider list module;
a business type user list module;
an invitation module; and
wherein the application is constructed and arranged to permit a physician to execute a physician's order via a multi-factor encrypted compliant digital authentication and to transmit the executed physician's order to a recipient on a network.

12. The physician order system of claim 11, wherein the user access module is constructed and arranged to permit one or more users to login to the application.

13. The physician order system of claim 11, wherein the user management module is constructed and arranged to permit an administrator to manage a plurality of user functions including at least one of dispatching invitations to users or creating user roles.

14. The physician order system of claim 11, wherein the user permission module is constructed and arranged to permit the administrator to control user permissions including at least one of viewing, modifying, or administering settings.

15. The physician order system of claim 11, wherein the data management module is constructed and arranged to store, catalog, organize, retrieve, and communicate information.

16. The physician order system of claim 11, further comprising a user registration module constructed and arranged to intake and process user registration information and permit an administrator to verify a submitted user registration.

17. A physician order system, comprising:
at least one computing device in operable connection with a network;
a memory that stores computer-executable components;
a processor that executes the computer-executable components stored in the memory, wherein the computer-executable components comprise:
a user registration module constructed and arranged to facilitate user type registration, identify unique users and prevent duplicate users, and notify an administrator of duplicate users;
a user management module constructed and arranged to allow an administrator to allow or deny user registration; and
a searchable data management module is constructed and arranged to store, catalog, organize, retrieve, and communicate information.

18. The physician order system of claim 17, further comprising:
a user permission module; and
wherein the user type may be at least one of a business types, healthcare provider, physician, or patient and wherein each user type is allowed permissions within the system as established by the user permission module.

19. The physician order system of claim 18, wherein the user permission module is constructed and arranged to permit business type users to upload files for signature by a physician or health care provider and healthcare providers and physician users are permitted to review, upload files, and execute orders.

20. The physician order system of claim 19, wherein the application is constructed and arranged to permit a physician to execute a physician's order via a multi-factor encrypted compliant digital authentication and to transmit the executed physician's order to a recipient on a network.
